(19) United States
(12) Patent Application Publication
(10) Pub. No.: US 2009/0024126 A1
(43) Pub. Date: Jan. 22, 2009

(54) Tissue fusion device

(76) Inventors: Ryan Artale, Boulder, CO (US); Barbara Bastian, Boulder, CO (US); Jeff Unger, Superior, CO (US)

Correspondence Address: Tyco Healthcare Group LP, 60 MIDDLETOWN AVENUE, NORTH HAVEN, CT, 06473, US

(21) Appl. No.: 11/880,021

(22) Filed: Jul. 19, 2007

(51) Int. Cl. A61B 18/18

(52) U.S. Cl. 606/51; 606/50

(57) ABSTRACT

A bipolar surgical instrument for fusing tissue includes first and second grasping members each having an end effector assembly attached at a distal end thereof. Each end effector assembly including a first jaw member with an active electrode disposed thereon and a second jaw member with a dielectric member disposed thereon. The first and second jaw members of each end effector are disposed in substantial opposing relation relative to one another and are movable from a first spaced position relative to one another to a second closer position for grasping tissue. Each active electrode is operably connected to an electrosurgical energy source. A surgical crimping tool is included and is selectively positionable to mechanically engage and crimp the active electrodes of the grasping members in a juxtaposed, side-by-side manner relative to one another prior to electrosurgical activation to fuse tissue into a unified tissue mass.

### BACKGROUND

The present disclosure relates to a method of fusing tissue utilizing RF energy and, more particularly, the present disclosure relates to a method of fusing tissue utilizing vessel or tissue sealing technology employing a unique combination of RF energy, pressure and gap distance to effectively seal or fuse tissue.

### TECHNICAL FIELD

During a large majority of operations, surgeons typically utilize sutures, clips and/or some other type of surgical fastener to hold adjacent tissue in opposition to promote tissue healing, graft two (or more) tissues together and/or perform an anastomosis between two tissue structures. In certain instances, biodegradable sutures are used, e.g., collagen “gut” sutures or synthetic polymer sutures, which have the added benefit of integrating with the body over time or dissolving thus eliminating many adverse reactions to the suture or “foreign body”.

Biological glues utilizing fibrin polymerization have also been used to provide a nontoxic, flowable material which sets into a solid to join tissue. However, these glues tend to have low adhesive strength and are more suitable for use as biological sealants which work in conjunction with other mechanical securement means, staples, sutures, etc. to join tissue.

Other techniques for tissue repair and tissue anastomosis have also been developed such as laser welding where a laser, e.g., ND:YAG, CO2, etc., applies light energy to thermally heat the tissue to a point where the tissue proteins denature and the collagenous elements of the tissue form a “biological glue” which adheres the tissue after the tissue area cools. However, the weakness of the weld joint is a primary disadvantage of laser welding, and various filler materials such as collagen must be introduced to improve the strength of the weld joint.

Laser welding is also a process whose success is dependent upon the proper management and control of many key properties which ultimately effect the overall success of fusing tissue. Some of these key properties include: the magnitude of the wavelength, energy level, absorption rate, and light intensity during irradiation and the concentration of the energy absorbing material. Moreover, laser welding is a relatively complex process which relies heavily on the use of energy-absorbing dyes with varying wavelengths and large and expensive laser units to thermally fuse tissue substances.

Vessel sealing or tissue fusion is a recently-developed technology which utilizes a unique combination of radiofrequency energy; pressure and gap control to effectively seal or fuse tissue between two opposing jaw members or sealing plates. “Vessel sealing” or “Tissue fusion” is defined as the process of liquefying the collagen, elastin and ground substances in the tissue so that it reforms into a fused mass with significantly-reduced demarcation between the opposing tissue structures.

In order to effectively “seal” or “fuse” tissue or vessels, two predominant mechanical parameters must be accurately controlled: 1) the pressure applied to the vessel or tissue; and 2) the gap distance between the conductive tissue contacting surfaces (electrodes). Accurate application of pressure is important for several reasons: to reduce the tissue impedance to a low enough value that allows enough electrosurgical energy through the tissue; to overcome the forces of expansion during tissue heating; and to contribute to the end tissue thickness which is an indication of a good seal. It has been determined that a good seal for certain tissues is optimum between 0.001 inches and 0.006 inches.

Typically, vessel sealing or tissue fusion is used for occluding vessels and tissue for subsequent resection. However, one envisioned application of vessel sealing or tissue fusion may be to effectively join tissue for tissue repair or grafting purposes (anastomosis, incision repair, vein or artery grafts) such as is discussed in commonly-owned, U.S. Pat. No. 7,147,638 entitled “ELECTROSURGICAL INSTRUMENT WHICH REDUCES THERMAL DAMAGE TO ADJACENT TISSUE” filed on Apr. 29, 2004, the entire contents of which are incorporated by reference herein.

### SUMMARY

The present disclosure relates to a bipolar surgical instrument for fusing tissue which includes first and second grasping members each having an end effector assembly disposed at a distal end thereof. Each end effector including a first jaw member with an active electrode disposed thereon and a second jaw member with a dielectric member disposed thereon. Each of the active electrodes is operably connected to an electrosurgical energy source. The active electrode of the first jaw member is energizable to a first electrical potential and the active electrode of the second jaw members is activatable to a second electrical potential. Alternatively, all of the electrodes or any combination thereof may be selectively energizable depending upon a particular purpose. As such, a separate return pad may be included to act as a return path to the generator.

The first and second jaw members are disposed in substantial opposing relation relative to one another and are movable from a first spaced position relative to one another to a second closer position for grasping tissue therebetween. A selectively positionable surgical crimping tool is also included which mechanically engages and crimps the active electrodes of the grasping members in a juxtaposed, side-by-side manner relative to one another prior to electrosurgical activation thereof.

In one embodiment, the jaw members engage tissue under a working pressure within the range of about 3 kg/cm² to about 16 kg/cm². In yet another embodiment, the crimping tool crimps the active electrodes of the grasping members under a working pressure within the range of about 3 kg/cm² to about 16 kg/cm² thereby squeezing exposed tissue between the juxtaposed active electrodes prior to electrosurgical activation of the active electrodes.

The active electrodes include an electrically conductive lateral side surface which substantially opposes a corresponding electrically conductive lateral side surface of the active electrode on the other grasping member. The lateral side surface of one (or more) of the active electrodes includes a or a plurality of stop members disposed thereon that is configured to maintain a gap distance between the corresponding electrically conductive lateral side surfaces. Preferably, the stop members are configured to maintain the gap distance to within a range of about 0.001 to about 0.010 inches.

The surgical crimping tool may be configured to apply pressure to both end effector assemblies in a direction normal or transverse to a longitudinal axis defined through the end effectors. In one envisioned embodiment, the surgical crimping tool is configured to apply pressure to both end effector assemblies in a direction normal and transverse to a longitudinal axis defined through the end effectors.

The present disclosure also relates to a bipolar surgical instrument for fusing tissue which includes first and second grasping members each having an end effector assembly attached at a distal end thereof. Each end effector includes a first jaw member with an active electrode disposed thereon and a second jaw member with a dielectric member (or other active electrode) disposed thereon. The first and second jaw members are disposed in substantial opposing relation relative to one another and are movable from a first spaced position relative to one another to a second closer position for grasping tissue therebetween. Each of the active electrodes is operably connected to an electrosurgical energy source and includes an electrically conductive lateral side surface which substantially opposes a corresponding electrically conductive lateral side surface of the active electrode on the other grasping member. The first jaw member is energizable to a first electrical potential and the active electrode of the second jaw members is activatable to a second electrical potential.

A surgical crimping tool is included which is selectively positionable to mechanically engage and crimp the active electrodes of the grasping members in a juxtaposed, side-by-side manner relative to one another prior to electrosurgical activation of the active electrodes. The surgical crimping tool crimps the active electrodes of the grasping members under a working pressure within the range of about 3 kg/cm² to about 16 kg/cm² thereby squeezing tissue between the juxtaposed active electrodes prior to electrosurgical activation thereof.

At least one stop member is disposed on at least one of the electrically conductive lateral side surfaces of at least one of the active electrodes. The stop member is configured to maintain a gap distance between the corresponding electrically conductive lateral side surfaces.

The present disclosure also relates to a method of fusing tissue using radiofrequency energy and includes the steps of: providing first and second grasping members each including an end effector assembly having a first jaw member with an active electrode disposed thereon and a second jaw member with a dielectric member disposed thereon. The first and second jaw members are disposed in substantial opposing relation relative to one another and operably connected to an electrosurgical energy source. The active electrode of the first jaw member is energizable to a first electrical potential and the active electrode of the second jaw members is activatable to a second electrical potential. A surgical crimping tool is also provided and is selectively positionable to mechanically engage and crimp the end effectors of the grasping members.

Alternatively, the method may include providing a first and second grasping members each including an end effector assembly having a first jaw member with an active electrode disposed thereon and a second jaw member with a second electrode disposed thereon. The electrodes may be activated in any foreseeable sequence to effect a particular surgical effect.

The method also includes the steps of: positioning the first and second jaw members of the first grasping member to grasp tissue therebetween leaving an inwardly exposed tissue end; positioning the first and second jaw members of the second grasping member to grasp tissue therebetween leaving an inwardly exposed tissue end; actuating the surgical crimping tool to crimp the end effectors of the first and second grasping members in a juxtaposed, side-by-side manner relative to one another to compress the exposed tissue ends against one another; and energizing the jaw members with radiofrequency energy to effectively fuse the exposed tissue ends.

### BRIEF DESCRIPTION OF THE DRAWINGS

Various embodiments of the subject methods and component parts associated therewith are described herein with reference to the drawings wherein:

FIG. 1 is a perspective view of a bipolar tissue fusion device according to one embodiment of the present disclosure;

FIG. 2A is a perspective view of a tissue grasping member for use with the bipolar tissue fusion device of FIG. 1;

FIG. 2B is an enlarged, perspective view of the area of detail of FIG. 2A.

FIG. 3A is an enlarged, perspective view of the crimping tool according to the present disclosure;

FIG. 3B is an enlarged, perspective view of a distal end of a crimping tool for use with the bipolar tissue fusion device of FIG. 1;

FIG. 4 is an enlarged schematic end view of the tissue grasping members engaged about tissue showing a fused tissue area between the tissue grasping members; and

FIGS. 5A-5C is a schematic illustration showing one method of fusing tissue according to the present disclosure including the steps of the tissue grasping members engaging tissue, the tissue ends being cut to facilitate fusing and the tissue ends being fused together; and

### DETAILED DESCRIPTION

The present invention relates to an apparatus and method for fusing tissues using so-called “vessel sealing” technology which involves a unique combination of radiofrequency (RF) energy, specified pressures and specific gap distances between opposing electrically conductive surfaces to effectively and consistently melt the tissue into a fused mass with limited demarcation. As mentioned above, vessel sealing utilizes a unique combination of controlled RF energy, pressure (within a specified pressure range) and specific gap distances between opposing tissue contacting surfaces to melt the two opposing surfaces into a unified fused mass. These parameters must be carefully controlled to assure consistent and effective sealing/fusion.

Heretofore, vessel sealing technology has been mainly used to effectively seal vessels and opposing tissue structures for subsequent separation or resection from the body. In other words a single vein or vessel is essentially sealed to reduce fluid flow therethrough and then resected and removed from the body. In other instances a large tissue structure is repeatably sealed and cut along the seal line and then resected and removed from the body.

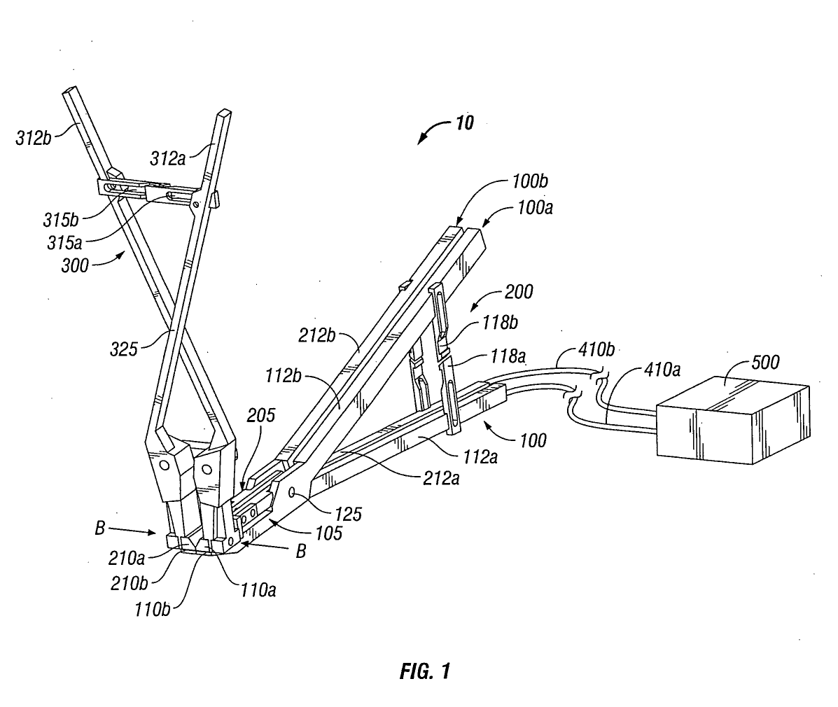

FIG. 1 shows one envisioned embodiment of a bipolar instrument which may be utilized to effectively fuse two tissue masses into a unified mass and is generally identified as forceps 10. Forceps 10 is envisioned for use with such surgical procedures as anastomosis, sealing skin incisions, vein or artery grafts, or any other surgical procedure where layers of tissue need to be fused together. For the purposes herein, either an endoscopic instrument or an open instrument may be utilized for fusing the tissue masses. Although the various figures generally show an open forceps design, obviously, different electrical and mechanical connections and considerations apply to each particular type of instrument, however, the novel aspects with respect to the forceps and its operating characteristics remain generally consistent with respect to both the open or endoscopic designs.

FIG. 1 shows forceps 10 which includes first and second grasping members 100a and 100b, respectively, each having an end effector assembly 105 and 205 disposed at a distal end thereof which mutually cooperate to grasp tissue for fusing purposes. Each tissue grasper 100a and 110b of the forceps 10 includes a cable lead 410a and 410b, respectively, which connects each grasper 100a and 100b to a source of electrosurgical energy, e.g., an electrosurgical generator 500.

First grasping member 100a includes first and second shafts 112a and 112b, respectively, having end effector assembly 105 at a distal end thereof. End effector assembly 105 includes upper and lower jaw members 110a and 110b which are selectively movable relative to one another about a pivot 125 from an open configuration wherein the jaw members 210a and 210b are spaced relative to one another to a second or closed position wherein the jaw members 110a and 110b cooperate to grasp tissue therebetween.

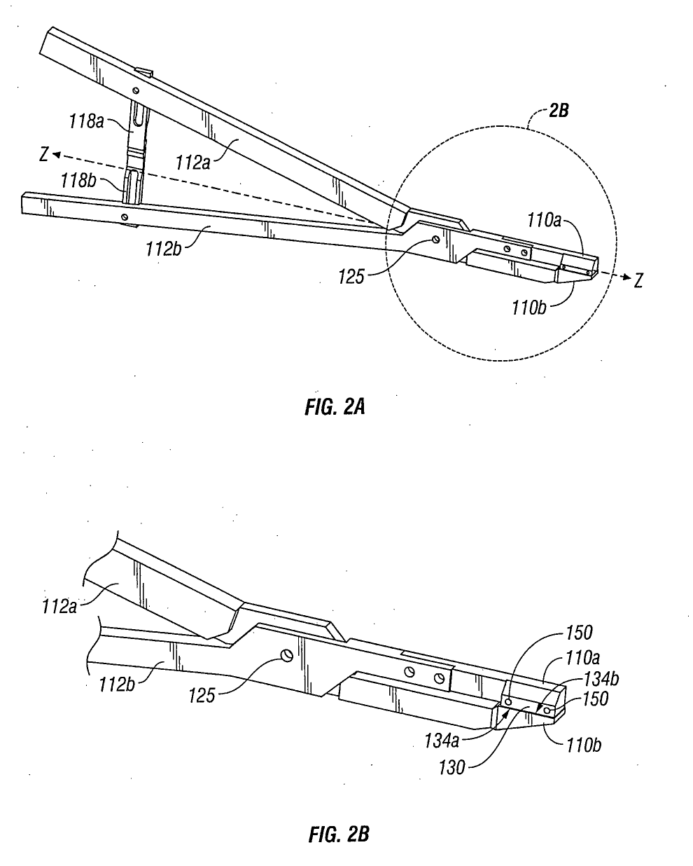

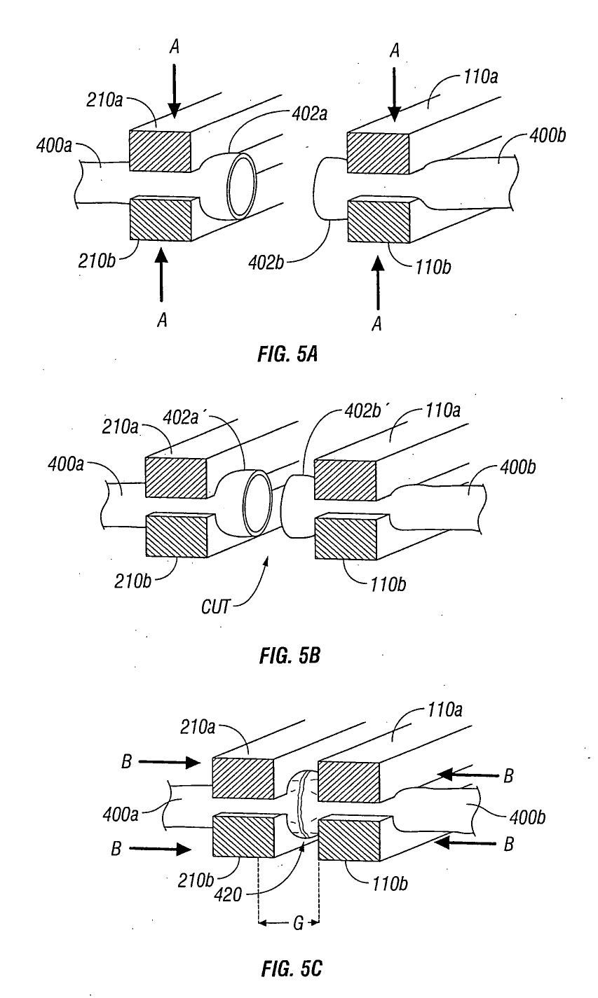

As best shown in FIG. 2B, upper jaw member 110a is typically conductive and includes an inwardly-facing tissue grasping surface 134a and a laterally-facing tissue sealing surface 130. Laterally-facing tissue sealing surface 130 includes one or more stop members 150 disposed thereon for maintaining a gap distance “G” (See FIG. 5C) between conductive surfaces 130 and 230 (described below) of the grasping members 110a and 110b as explained in more detail below. Lower jaw member 110b is typically dielectric or insulative and includes a tissue grasping surface 134b which opposes tissue grasping surface 134a.

As best shown in FIG. 2A first grasping member 100a is movable about pivot 125 to initially grasp tissue between jaw members 110a and 110b. A pair of mechanically interengaging elements 118a and 118b are disposed between the shafts 112a and 112b and are configured to lock the jaw members 110a and 110b in an engaged position about tissue. It is envisioned that the interengaging elements 118a and 118b may be configured to maintain a predetermined clamping pressure between the jaw members 110a and 110b to facilitate sealing of tissue as will be explained in more detail below.

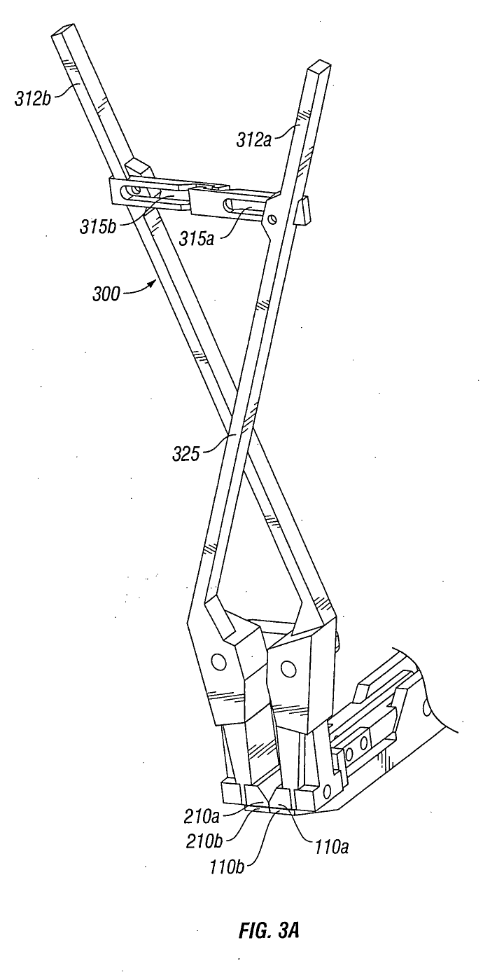

Grasping member 100b includes similar elements to grasping member 100a. More particularly, second grasping member 100b includes first and second shafts 212a and 212b, respectively, having end effector assembly 205 at a distal end thereof. End effector assembly 205 includes upper and lower jaw members 210a and 210b which are selectively movable relative to one another about a pivot 225 (See FIG. 3B) from an open configuration wherein the jaw members 210a and 210b are spaced relative to one another to a second or closed position wherein the jaw members 210a and 210b cooperate to grasp tissue therebetween. As best shown in FIG. 3B and much like upper jaw member 110a, upper jaw member 210a is conductive and includes an inwardly-facing tissue grasping surface and a laterally-facing tissue sealing surface 130 which opposes tissue sealing surface 130.

As best shown in FIGS. 1 and 3, the forceps 10 also includes a surgical crimping tool 300 which is configured to crimp or squeeze the respective jaw members 110a, 110b and 210a, 210b of the tissue grasping members 100a and 100b in a lateral direction (as referenced by arrow “B”) relative to a longitudinal axis “Z” defined between the end effector assemblies 105 and 205. More particularly, the crimping tool 300 includes a pair of shaft members 312a and 312b each having a crimping head 310a and 310b, respectively, attached to a distal end thereof. The shaft members 312a and 312b are rotatable in a scissor-like fashion about a common pivot 325 to move the crimping heads 310a and 310b from a spaced position relative to the end effectors 105 and 205 to a crimping position wherein the crimping heads engage the end effectors 105 and 205.

As best shown in FIG. 3B, the crimping heads 310a and 310b are configured to securely engage an outer periphery of a respective end effector assembly 105 and 205 of one of the grasping members 100a and 100b for crimping purposes. More particularly, the crimping heads 310a and 310b include insulative members 311a and 311b, respectively, disposed at an inner periphery of each crimping head 310a and 310b which are configured to mechanically engage the conductive jaw members 110a and 210a of each grasping member 110 and 100b. Steps 314a and 314b are included at the distal ends of the insulative portions 311a and 311b of the crimping heads 310a and 310b, respectively, and are configured to facilitate mechanical engagement of the conductive jaw members 110a and 210a with the crimping heads 310a and 310b. It is envisioned that the crimping heads 310a and 310b are configured to apply lateral crimping pressure to the conductive jaw members 110a and 210a which are typically made from a hardened conductive material such as stainless steel, aluminum and the like.

Each shaft member 312a and 312b of the crimping tool 300 includes a mechanical interface 315a and 315b which interengage one another on respective shaft members 312a and 312b to secure the crimping tool 300 in a crimped position for electrosurgical activation. The interfaces 315a and 315b may be configured to hold and maintain a specific strain energy on the shaft members 312a and 312b to provide a particular crimping force to the crimping heads 310a and 310b. For example, it is envisioned that a magnitude of pressure exerted on the tissue sealing surfaces 130 and 230 by the crimping heads 310a and 310b is important in assuring proper surgical fusion of the two tissue structures.

Pressures within a working range of about 3 kg/cm² to about 16 kg/cm² and, preferably, within a working range of 4.5 kg/cm² to 8.5 kg/cm² have been shown to be effective for fusing various tissue types. In addition to keeping the pressure within a working range (i.e., about 3 kg/cm² to about 16 kg/cm²) and the gap distance “G” within a specified range (i.e., about 0.001 inches to about 0.010 inches) the electrical power should be kept within the range of about 1 W to about 350 W, about 1 Vrms to about 400 Vrms and about 0 Amps to about 5.5 Amps. Moreover, the tissue sealing surfaces 130 and 230 should be designed for low thermal mass to optimize thermal heating between jaw members 110a and 210a and minimize thermal loss through the device.

As mentioned above, one or both tissue sealing surfaces 130 and 230 include one or more stop members 150 disposed thereon for maintaining a gap distance “G” (See FIG. 5C) between the sealing surfaces 130 and 230 of the grasping members 110 and 100b. The stop member(s) 150 extends from the sealing surface 130, 230 a predetermined distance according to the specific material properties of the stop members 150 (e.g., compressive strength, thermal expansion, etc.) to yield a consistent and accurate gap distance “G” within the above specified ranges during the fusion process. Stop members 150-150 may be made from an insulative material, e.g., parylene, nylon and/or ceramic, and dimensioned to limit opposing movement of the jaw members 110a, 110b and 210a, 210b to within the above-mentioned gap range. Stop members 150 can be disposed on one or both of the jaw members 110a and 210b and 420 and may be dimensioned in a variety of different shapes and sizes, longitudinal, circular, ridge-like, etc. Many different stop member configurations are envisioned such as those configurations described in U.S. application Ser. No. 10/471,818 entitled “VESSEL SEALER AND DIVIDER WITH NON-CONDUCTIVE STOP MEMBERS”, the entire contents of which are incorporated by reference herein.

The jaw members 410 and 420 are electrically isolated from one another such that electrosurgical energy can be effectively transferred to electrically conductive tissue surfaces 130 and 230 and through the tissue to fuse the tissue together into a unified mass.

As best shown in the schematic representation of FIGS. 5A-5C, the end effector assembly 105 and 205 of each grasping member 100a and 100b is positioned about a tissue structure 400a and 400b leaving an exposed inwardly extending tissue end 402a and 402b, respectively. The grasping members 100a and 100b are then actuated to close the respective jaw members 110a, 110b and 210a, 210b in the direction of arrows “A” about the tissue 400a and 400b, respectively. The exposed tissue ends are then cut to form a clean fusing edge 402a′ and 402b′. It is envisioned that the tissue halves 400a and 400b may be cut prior to grasping the tissue halves between the jaw members 110a, 110b and 210a, 210b.

Once the tissue 400a and 400b is grasped between the jaw members 110a, 110b and 210a, 210b, respectively, the crimping tool 300 is positioned to engage the conductive jaw members (See FIG. 3B) and actuated to force the tissue ends 402a and 402b inwardly against one another within the above working pressure range of about 3 kg/cm² to about 16 kg/cm². As mentioned above, once crimped, the stop members 150 maintain a gap distance “G” between the conductive surfaces 130 and 230 between about 0.001 inches to about 0.010 inches.

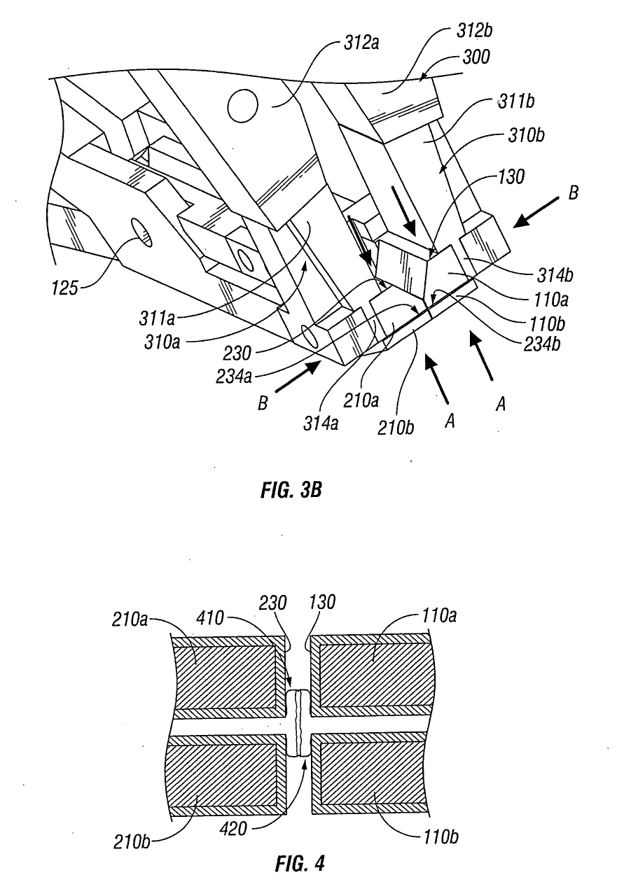

By controlling the intensity, frequency and duration of the RF energy applied to the active jaw members 110a and 210a, the user can selectively fuse the two tissue ends 402a and 402b as shown in FIGS. 4 and 5C to create a fused tissue line 420. Once fused, the user uncrimps the crimping tool 300 and disengages the tissue grasping members 100a and 100b to release the tissue. As can be appreciated, the forceps 10 may be used to seal incisions, form an anastomosis between two tissue structures or vessels, skin grafts, artery or vein grafts, etc.

It is envisioned that the above forceps 10 may be utilized in connection with a closed-loop RF control system which optimizes fusion based upon pre-surgical conditions or changes in physical or electrical conditions during the fusion process. One example of a closed-loop control system is described in commonly-owned and concurrently-filed U.S. Pat. No. 7,137,980 entitled “METHOD AND SYSTEM FOR CONTROLLING OUTPUT OF RF MEDICAL GENERATOR” and commonly-owned and concurrently-filed U.S. patent application Ser. No. 10/835,657 entitled “METHOD AND SYSTEM FOR PROGRAMMING AND CONTROLLING AN ELECTROSURGICAL GENERATOR SYSTEM” which is incorporated in its entirety by reference herein. In general, the closed-loop control, system includes a user interface for allowing a user to select at least one pre-surgical parameter, such as the type of surgical instrument operatively connected to the generator, the type of tissue and/or a desired surgical effect. A sensor module may also be included for continually sensing at least one of electrical and physical properties proximate the surgical site and generating at least one signal relating thereto.

The closed loop control system also includes a control module for continually receiving or monitoring surgical parameters and each of the signals from the sensor module and processing each of the signals in accordance with a desired surgical effect using a microprocessor, computer algorithm and/or a look-up table. The control module generates at least one corresponding control signal relating to each signal from the sensor module, and relays the control signal to the electrosurgical generator for controlling the generator. The closed loop system may be employed in a feedback circuit or part of a surgical method for optimizing a surgical seal. The various methods described herein may also include the steps of: applying a series of electrical pulses to the surgical site; continually sensing electrical and physical properties proximate the surgical site; and varying pulse parameters of the individual pulses of the series of pulses in accordance with the continually-sensed properties.

A controller (not shown) may also be electrically interposed between the generator 500 and the conductive jaw members 110a and 210a to regulate the RF energy supplied thereto depending upon certain electrical parameters, i.e., current impedance, temperature, voltage, etc. For example, the forceps 10 or the controller may include one or more smart sensors (not shown) which communicate with the electrosurgical generator 500 (or smart circuit, computer, feedback loop, etc.) to automatically regulate the electrical intensity (waveform, current, voltage, etc.) to enhance the fusing process. The sensor may measure or monitor one or more of the following parameters: temperature, impedance, change in impedance over time and/or changes in the power or current applied over time. An audible or visual feedback monitor (not shown) may be employed to convey information to the surgeon regarding the overall fusion quality or the completion of an effective fusion between the tow tissue structures. Examples of a various control circuits, generators and algorithms which may be utilized are disclosed in commonly-owned U.S. Pat. No. 6,228,080 and U.S. application Ser. No. 10/073,761 entitled “VESSEL SEALING SYSTEM” the entire contents of both of which are hereby incorporated by reference herein.

From the foregoing and with reference to the various figure drawings, those skilled in the art will appreciate that certain modifications can also be made to the present disclosure without departing from the scope of the present disclosure. For example, the RF energy may need to be regulated or controlled (feedback loop, algorithm, closed loop system, etc.) depending upon the type of tissue being fused. It is envisioned that various sensors may be employed to closely monitor various tissue parameters (impedance, temperature, moisture, etc.) to optimize the fusion process for each type of tissue.

It is also envisioned that the forceps 10 may be designed such that it is fully or partially disposable depending upon a particular purpose or to achieve a particular result. For example, end effector assemblies 105 and 205 may be selectively and releasably engageable with the distal end of the respective shaft 112a, 112b and 212a, 212b. In this instance, the forceps 10 would be considered “partially disposable” or “reposable”, i.e., a new or different end effector assembly 105, 205 selectively replaces the old end effector assembly as needed. The crimping tool 300 may also be reposable and include insulative inserts 311a and 311b which may be readily exchanged after each surgery. Various types of mechanical interfaces (not shown) may be utilized to facilitate replacement of the insulative inserts as needed, e.g., snap-fit, slide-fit, etc.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A bipolar surgical instrument for fusing tissue, comprising:
first and second grasping members, each of the grasping members including an end effector assembly having a first jaw member with an active electrode disposed thereon and a second jaw member with a dielectric member disposed thereon, the first and second jaw members being in substantial opposing relation relative to one another and being movable from a first spaced position relative to one another to a second closer position for grasping tissue therebetween, each of the active electrodes being operably connected to an electrosurgical energy source; and
a surgical crimping tool selectively positionable to mechanically engage and crimp the active electrodes of the grasping members in a juxtaposed, side-by-side manner relative to one another prior to electrosurgical activation thereof.

2. A bipolar surgical instrument according to claim 1 wherein the active electrode of the first jaw member is energizable to a first electrical potential and the active electrode of the second jaw members is activatable to a second electrical potential.

3. A bipolar surgical instrument according to claim 1 wherein the jaw members engage tissue under a working pressure within the range of about 3 kg/cm² to about 16 kg/cm².

4. A bipolar surgical instrument according to claim 1 wherein the surgical crimping tool crimps the active electrodes of the grasping members under a working pressure within the range of about 3 kg/cm² to about 16 kg/cm² thereby squeezing exposed tissue between the juxtaposed active electrodes prior to electrosurgical activation of the active electrodes.

5. A bipolar surgical instrument according to claim 1 wherein each of the active electrodes includes an electrically conductive lateral side surface which substantially opposes a corresponding electrically conductive lateral side surface of the active electrode on the other grasping member.

6. A bipolar surgical instrument according to claim 4 wherein the lateral side surface of at least one active electrode includes at least one stop member disposed thereon which is configured to maintain a gap distance between the corresponding electrically conductive lateral side surfaces.

7. A bipolar surgical instrument according to claim 6 wherein the stop members are configured to maintain the gap distance to within a range of about 0.001 to about 0.010 inches.

8. A bipolar surgical instrument according to claim 1 wherein the surgical crimping tool is configured to apply pressure to both end effector assemblies in a direction normal to a longitudinal axis defined through the end effectors.

9. A bipolar surgical instrument according to claim 1 wherein the surgical crimping tool is configured to apply pressure to both end effector assemblies in a direction transverse to a longitudinal axis defined through the end effectors.

10. A bipolar surgical instrument according to claim 1 wherein the surgical crimping tool is configured to apply pressure to both end effector assemblies in a direction normal to a longitudinal axis defined through the end effectors and transverse to the longitudinal axis defined through the end effectors.

11. A bipolar surgical instrument for fusing tissue, comprising:
first and second grasping members, each of the grasping members including an end effector assembly having a first jaw member with an active electrode disposed thereon and a second jaw member with a dielectric member disposed thereon, the first and second jaw members being in substantial opposing relation relative to one another and movable from a first spaced position relative to one another to a second closer position for grasping tissue, each of the active electrodes includes an electrically conductive lateral side surface which substantially opposes a corresponding electrically conductive lateral side surface of the active electrode on the other grasping member, each of the active electrodes being operably connected to an electrosurgical energy source;
a surgical crimping tool selectively positionable to mechanically engage and crimp the active electrodes of the end effector assemblies of the grasping members in juxtaposed, side-by-side manner relative to one another prior to electrosurgical activation of the active electrodes, wherein the surgical crimping tool crimps the active electrodes of the grasping members under a working pressure within the range of about 3 kg/cm² to about 16 kg/cm² thereby squeezing tissue between the juxtaposed active electrodes prior to electrosurgical activation thereof; and
at least one stop member disposed on at least one of the electrically conductive lateral side surfaces of at least one of the active electrodes, the stop member being configured to maintain a gap distance between the corresponding electrically conductive lateral side surfaces.

12. A bipolar surgical instrument according to claim 11 wherein the active electrode of the first jaw member is energizable to a first electrical potential and the active electrode of the second jaw members is activatable to a second electrical potential.

13. A bipolar surgical instrument according to claim 11 wherein the stop members are configured to maintain the gap distance to within a range of about 0.001 to about 0.010 inches.

14. A method of fusing tissue using radiofrequency energy, comprising the steps of:
providing:
first and second grasping members, each of the grasping members including an end effector assembly having a first jaw member with an active electrode disposed thereon and a second jaw member with a dielectric member disposed thereon, the first and second jaw members being in substantial opposing relation relative to one another, each of active electrodes being operably connected to an electrosurgical energy source;
a surgical crimping tool selectively positionable to mechanically engage and crimp the end effectors of the grasping members;
positioning the first and second jaw members of the first grasping member to grasp tissue therebetween leaving an inwardly exposed tissue end;
positioning the first and second jaw members of the second grasping member to grasp tissue therebetween leaving an inwardly exposed tissue end;
actuating the surgical crimping tool to crimp the end effectors of the first and second grasping members in a juxtaposed, side-by-side manner relative to one another to compress the exposed tissue ends against one another; and
energizing the jaw members with radiofrequency energy to effectively fuse the exposed tissue ends.

15. A method of fusing tissue using radiofrequency energy according to claim 14 wherein the active electrode of the first jaw member is energizable to a first electrical potential and the active electrode of the second jaw members is activatable to a second electrical potential.

16. A method of fusing tissue using radiofrequency energy according to claim 14 wherein the exposed tissue ends are compressed under a working pressure within the range of about 3 kg/cm² to about 16 kg/cm².

17. A method of fusing tissue using radiofrequency energy according to claim 14 wherein each of the active electrodes of the providing step includes an electrically conductive lateral side surface which substantially opposes a corresponding electrically conductive lateral side surface of the active electrode on the other grasping member.

18. A method of fusing tissue using radiofrequency energy according to claim 16 wherein the lateral side surface of at least one active electrode includes at least one stop member disposed thereon which is configured to maintain a gap distance between the corresponding electrically conductive lateral side surfaces.

19. A method of fusing tissue using radiofrequency energy according to claim 17 wherein the stop members are configured to maintain the gap distance to within a range of about 0.001 to about 0.010 inches.
